(19) United States
(12) Patent Application Publication
(10) Pub. No.: US 2024/0209344 A1
(43) Pub. Date: Jun. 27, 2024

(54) DEVICE AND METHOD FOR PHOTOACTIVATION OF PLATELET-RICH PLASMA

(71) Applicant: Lance Liberti, Tampa, FL (US)

(72) Inventor: Lance Liberti, Tampa, FL (US)

(21) Appl. No.: 18/097,463

(22) Filed: Jan. 16, 2023

Related U.S. Application Data
(60) Provisional application No. 63/435,133, filed on Dec. 23, 2022.

(51) Int. Cl. C12N 13/00; C12M 1/42; C12N 5/078

(52) U.S. Cl. CPC C12N 13/00; C12M 35/00; C12N 5/0644

(57) ABSTRACT

A device and method for activating platelet-rich plasma prior to injection. The device includes a lamp that has multiple LEDs for generating a desired light spectrum, such as red, blue, yellow, and green. The PPR is activated by exposure to polychromatic light generated by the lamp.

### CROSS-REFERENCE TO RELATED APPLICATIONS

This application claims priority from a U.S. provisional patent application Ser. No. 63/435,133, filed on Dec. 23, 2022, which is incorporated herein by reference in its entirety.

### FIELD OF INVENTION

The present invention relates to a device and method for the activation of Platelet-Rich Plasma, and more particularly, the present invention related to a device and method for photoactivation of PRP using polychromatic light.

### BACKGROUND

It has been widely accepted that platelet-Rich Plasma, having the platelets activated, can stimulate cellular angiogenesis at a greater rate. The activation of platelet, typically, refers to the processes initiated during PRP activation. The processes include degranulation releasing growth factors from the alpha granules of the platelets, and the fibrin cascade which forms clots to stop bleeding and trap the molecules at the site of injury. The activation process is a necessity for PRP to achieve maximum therapeutic benefit in many clinical applications.

Different processes for the activation of PRP are known in the art. However, the known method generally requires the addition of certain chemical and/or hormone additives to the plasma samples. Such methods have many drawbacks and limitations, which result in a decrease in efficiency and side effects in the patients. Moreover, the addition of chemicals and/or hormones requires standardization and optimization studies which make the process costly. The addition of chemicals and/or hormones by estimation and personal experiences leads to undesirable results. Moreover, the use of chemicals and hormones may require regulatory approvals which are often not readily available.

A need is therefore appreciated for a novel method of activating platelets in PRP without chemicals and/or hormones.

### SUMMARY OF THE INVENTION

The following presents a simplified summary of one or more embodiments of the present invention to provide a basic understanding of such embodiments. This summary is not an extensive overview of all contemplated embodiments and is intended to neither identify key or critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to a device and method for activating platelets in a PRP sample without chemicals or hormones.

It is another object of the present invention that the device and method provide for safer activation of the platelets.

It is still another object of the undesirable side effects associated with the use of chemicals and hormones that are avoided.

It is yet another object of the present invention that the device and method can be easily used in clinical settings.

It is a further object of the present invention that any special technical skills are not required for operating the disclosed device.

It is still a further object of the present invention that the activation of platelets helps growth factor release from the platelets.

It is yet a further object of the present invention that the activation of platelets increases pro-inflammatory cytokine receptors, IL 1 Ra and IL2 Ra, and beta-endorphin.

In one aspect, disclosed is a device and method for activating platelets in a PRP sample taken from a patient, by exposing the PRP sample to polychromatic light before injection.

In one aspect, disclosed is a device for activating platelet-rich plasma, the device comprises a housing; one or more receptacles in the housing, wherein the one or more receptacles are configured to receive one or more containers; and at least one lamp configured to generate a polychromatic light in a predetermined light spectrum, wherein the at least one lamp is positioned to expose a portion of the one or more containers in the one or more receptacles to the polychromatic light. The lamp comprises a combination of red, blue, orange, and yellow lights. The lamp has ten red LEDs, ten yellow LEDs, ten green LEDs, and ten orange LEDs. The device further comprises a chamber encased within the housing, wherein the at least one lamp protrudes into the chamber, and the one or more receptacles are provided in the chamber, wherein an inner surface of the chamber has a reflective coating for evenly exposing the one or more containers to the polychromatic light. The device further comprises a timer to set a duration for polychromatic light exposure. The device further comprises one or more control buttons and one or more displays, the one or more control buttons are configured to set the duration of keeping the at least one lamp turned on.

In one aspect, disclosed is a method for activating platelet-rich plasma, the method comprises: providing a device comprising: a housing, one or more receptacles in the housing, wherein the one or more receptacles are configured to receive one or more containers, and at least one lamp configured to generate a polychromatic light in a predetermined light spectrum, wherein the at least one lamp is positioned to expose a portion of the one or more containers in the one or more receptacles to the polychromatic light; collecting a platelet-rich plasma sample in a container; placing the container in a receptacle of the one or more receptacles; and exposing the plate-rich plasma sample in the container to the polychromatic light for a pre-determined duration.

In one aspect, disclosed is a method for activating platelet-rich plasma, the method comprises exposing platelet-rich plasma samples to polychromatic light for a predetermined duration.

### BRIEF DESCRIPTION OF THE DRAWINGS

The accompanying figures, which are incorporated herein, form part of the specification and illustrate embodiments of the present invention. Together with the description, the figures further explain the principles of the present invention and to enable a person skilled in the relevant arts to make and use the invention.

FIG. 1 is a front view of the device, according to an exemplary embodiment of the present invention.

FIG. 2 is a rear view of the device, according to an exemplary embodiment of the present invention.

### DETAILED DESCRIPTION

Subject matter will now be described more fully hereinafter with reference to the accompanying drawings, which form a part hereof, and which show, by way of illustration, specific exemplary embodiments. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is to describe particular embodiments only and is not intended to be limiting of embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely to illustrate the general principles of the invention since the scope of the invention will be best defined by the allowed claims of any resulting patent.

Disclosed is a device and method for activating platelet-rich plasma (PRP) safely and efficiently. The disclosed method includes exposing the PRP to polychromatic light for 10 minutes before injection. The disclosed method allows activating platelets in a PRP sample in a safe, autologous, and cost-effected manner.

The disclosed device includes a lamp for exposing the PRP to polychromatic light. The lamp includes a plurality of lights in different wavelength ranges to cover a desired light spectrum. The disclosed number of such lights required can be optimized by suitable calculations. In one implementation, the lamp can include 40 lights that include a combination of ten red lights, ten blue lights, ten green lights, and ten yellow lights. The device further includes a power unit to power said lamp. A timer can also be provided to automatically turn off the lamp after pre-set duration.

In one implementation, the disclosed device can also include a controller that can be configured to select the types and number of lights to be turned on for desired light spectrum. The duration of keeping the combination of lights turned on can also be configured in the controller. The controller includes a microcontroller or any other processing circuitry.

In one implementation, the lamp can include LED lights, however, any other light is within the scope of the present invention.

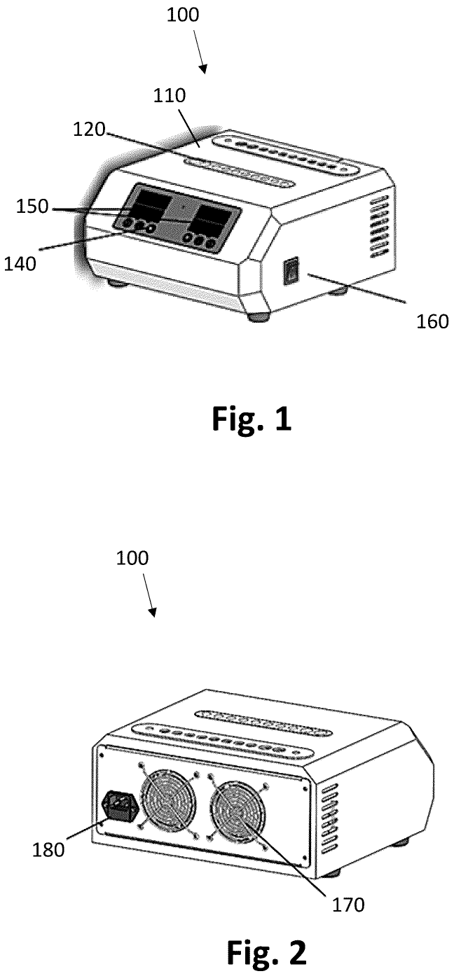

Referring to FIGS. 1 and 2 which show an exemplary embodiment of the disclosed device. The device 100 includes a housing 110 that encases the lamp and the controller. The timer can be inbuilt into the controller. Or the timer can be a separate module encased within the housing. It is understood that the controller and the timer can be built onto a printed circuit board (PCB) and the lamp can be operably connected to the PCB. As shown in FIG. 1, several receptacles in the housing can receive syringes. The PRP sample can be taken in a syringe, and the same syringe can be placed into a receptacle 120. Multiple receptacles can be provided to simultaneously expose multiple syringes to the polychromatic light. The housing can encase a chamber into which the syringes protrude, and the lamp can be provided to illuminate the chamber, and thus the syringes. A suitable reflective coating can be provided in the chamber to ensure that the PRP sample in the syringe is evenly exposed to the light from the lamp.

In certain implementations, more than one lamp can be used to evenly expose the PRP sample in the syringe to the polychromatic light. In certain implementations, two chambers can be provided in the housing, wherein each chamber has a lamp. Half of the receptacles can be provided in one chamber and the rest of the receptacles can be provided in the second chamber. Lamps in the two chambers can be independently operated. FIG. 1 shows two control panels 150 for the two chambers and the two lamps, and control buttons 140 are also shown for each control panel.

It is understood, however, that instead of a syringe, any suitable container can also be used. Moreover, the depth of the receptacles can be such that the PRP sample in the syringe/container can be fully and evenly exposed to the polychromatic light from the lamp.

On side of the housing in FIG. 1 is shown a power switch 160 that can be used to turn on and off the disclosed device. FIG. 2 shows the rear side of the device. A power socket 180 can be seen for connecting to an electric cord and the power supply. The device can further include suitable fans 170 for dissipating any heat generated by the lamps and the controller. Two fans are shown in FIG. 2 for the two chambers and two lamps. It may be noted that the temperature within the chambers can be controlled within predefined limits to prevent exposure of the PRP to excessive or undesired heat levels.

The disclosed method includes steps of collecting PRP in a syringe or a container. Turning on the disclosed device by using a switch of the disclosed device or switch of the power supply. The syringe or the container can then be placed into a receptacle of the disclosed device. It is understood that the syringe can also be placed in the receptacle before turning the device on. The timer can be set for the desired duration, such as for 10 minutes. The controller/timer of the disclosed device can turn off the device after a pre-set duration and make a beep to inform the user.

In one implementation, an option to select a desired light spectrum can also be provided. Suitable control buttons operably coupled to the controller can be provided.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A device for activating platelet-rich plasma, the device comprises:
a housing;
one or more receptacles in the housing, wherein the one or more receptacles are configured to receive one or more containers; and
at least one lamp configured to generate a polychromatic light in a predetermined light spectrum, wherein the at least one lamp is positioned to expose a portion of the one or more containers in the one or more receptacles to the polychromatic light.

2. The device according to claim 1, wherein the lamp comprises a combination of red, blue, orange, and yellow lights.

3. The device according to claim 2, wherein the lamp has ten red LEDs, ten yellow LEDs, ten green LEDs, and ten orange LEDs.

4. The device according to claim 1, wherein the device further comprises a chamber encased within the housing, wherein the at least one lamp protrudes into the chamber, and the one or more receptacles are provided in the chamber, wherein an inner surface of the chamber has a reflective coating for evenly exposing the one or more containers to the polychromatic light.

5. The device according to claim 1, wherein the device further comprises a timer to set a duration for polychromatic light exposure.

6. The device according to claim 1, wherein the device further comprises one or more control buttons and one or more displays, the one or more control buttons are configured to set the duration of keeping the at least one lamp turned on.

7. A method for activating platelet-rich plasma, the method comprises:
providing a device comprising:
a housing,
one or more receptacles in the housing, wherein the one or more receptacles are configured to receive one or more containers, and
at least one lamp configured to generate a polychromatic light in a predetermined light spectrum, wherein the at least one lamp is positioned to expose a portion of the one or more containers in the one or more receptacles to the polychromatic light;
collecting a platelet-rich plasma sample in a container;
placing the container in a receptacle of the one or more receptacles; and
exposing the plate-rich plasma sample in the container to the polychromatic light for a pre-determined duration.

8. The method according to claim 7, wherein the lamp comprises a combination of red, blue, orange, and yellow lights.

9. The method according to claim 8, wherein the lamp has ten red LEDs, ten yellow LEDs, ten green LEDs, and ten orange LEDs.

10. The method according to claim 7, wherein the device further comprises a chamber encased within the housing, wherein the at least one lamp protrudes into the chamber, and the one or more receptacles are provided in the chamber, wherein an inner surface of the chamber has a reflective coating for evenly exposing the one or more containers to the polychromatic light.

11. The method according to claim 7, wherein the device further comprises a timer to set a duration of polychromatic light exposure.

12. The method according to claim 7, wherein the device further comprises one or more control buttons and one or more displays, the one or more control buttons are configured to set a duration of keeping the at least one lamp turned on.

13. A method for activating platelet-rich plasma, the method comprises:
exposing platelet-rich plasma samples to polychromatic light for a predetermined duration.
